(19) United States
(12) Patent Application Publication
(10) Pub. No.: US 2005/0165438 A1
(43) Pub. Date: Jul. 28, 2005

(54) Device and method for introducing surgical instruments into the body

(76) Inventor: Vadim Gritsus, Rockaway, NJ (US)

Correspondence Address: FRISHAUF, HOLTZ, GOODMAN & CHICK, PC, 220 5TH AVE. FL 18, NEW YORK, NY, 10001-7708, US

(21) Appl. No.: 10/851,857

(22) Filed: May 21, 2004

Related U.S. Application Data
(60) Provisional application No. 60/538,940, filed on Jan. 23, 2004.

(51) Int. Cl. A61B 17/00

(52) U.S. Cl. 606/190

(57) ABSTRACT

A device for facilitating insertion of a surgical instrument into a human body through an opening in a body wall including a cone-shaped member having a rounded front end, an internal cavity extending inward from a rear edge and a through opening arranged at the front end. At least part of the surgical instrument is received in the cavity such that the surgical instrument is insertable into the body through the opening in the body wall by urging the member through the opening with its front end forward and thereby causing tissue surrounding the opening to spread outward as the member passes through the body wall. The instrument is separated from the member after insertion of the member and the instrument into the body. The member can be removed from the body through same opening through which it was inserted into the body.

### CROSS-REFERENCE TO RELATED APPLICATION

This application claims priority under 35 U.S.C. §119(e) of U.S. provisional patent application Ser. No. 60/538,940 filed Jan. 23, 2004, the entire disclosure of which is incorporated by reference herein.

### FIELD OF THE INVENTION

The present invention relates generally to devices and methods for enabling the percutaneous introduction of surgical instruments into the human body and more particularly to devices and methods for introducing blunt surgical instruments, such as a surgical circular stapler or stapler anvil, through the body wall, for example, during intracorporeal creation of a surgical anastomoses in laparoscopic surgical procedures.

### BACKGROUND OF THE INVENTION

In a variety of surgical procedures, it is necessary to use a surgical stapler to staple separated internal parts of the body together. For example, in some laparoscopic surgical procedures, it is necessary to create an anastomosis by joining, e.g., separated parts of the intestines, and this is usually accomplished by stapling the separated parts together by means of a surgical stapler.

During such a laparoscopic procedure, it is difficult to insert the surgical stapler and/or stapler anvil through the laparoscopic incision or opening made in the abdominal or body wall in order to enable the stapler or anvil to be subsequently manipulated to the area in which it is needed to staple the body parts together. In particular, since the size of surgical staplers and anvils varies, the laparoscopic incision might not be large enough for the particular stapler or anvil being used. Moreover, common staplers and anvils usually have a blunt forward end which makes it difficult to introduce them easily through the opening in the body wall.

An additional problem with the insertion of staplers through an opening in the body wall is that fragments of the tissues around the opening in the body wall can become lodged in the open end of the stapler and cause a subsequent malfunction of the stapling mechanism in the stapler.

### OBJECTS AND SUMMARY OF THE INVENTION

Accordingly, it is an object of the present invention to provide a new device and method for facilitating the insertion of a surgical instrument into the body through an opening in the body wall. The invention is particularly useful for facilitating the insertion of blunt surgical instruments, such as a surgical stapler or stapler anvil, into the body through an opening in the body wall.

It is another object of the present invention to provide a new device and method for facilitating the passage of a surgical instrument through a small opening in the abdominal wall which temporarily spreads the tissues around the opening during passage of the surgical instrument therethrough. In this manner, a surgical instrument which is larger than the size of the opening can be effectively inserted through the opening.

In order to achieve the above objects and other objects, a device for facilitating insertion of a surgical instrument into a human body through an opening in a body wall in accordance with the invention includes a cone-shaped member having a rounded front end, an internal cavity extending inward from a rear edge and a through opening arranged at the front end. The cavity is dimensioned such that at least a part of the surgical instrument is receivable therein. During use, the surgical instrument, after insertion into the cavity of the member, is inserted into the body through the opening in the body wall by urging the member through the opening with its front end forward and thereby causing tissue surrounding the opening to spread outward as the member passes through the body wall. The instrument is separated from the member after insertion of the member and the instrument into the body. The member and/or instrument can be removed from the body through same opening through which it was inserted into the body.

The member and cavity can be dimensioned to accommodate different surgical instruments, including but not limited to circular surgical staplers and stapler anvils.

The through hole is used to enable removal of the member from the body once the instrument is situated in the body. This is achieved by inserting a suture thread, or any human-compatible thread, through the through opening and tying its ends together to form a loop. A part of the loop is maintained outside of the body during the insertion of the member into the body, and then after the instrument is removed from the cavity, the thread outside of the body is grasped and pulled to remove the member from the body. The member is preferably removed from the body with its front end forward.

A method for introducing a surgical instrument into a body through an opening in a body wall in accordance with the invention entails inserting at least a part of the instrument into a cavity formed in a rear of a cone-shaped member having a rounded front end, inserting the member with the instrument at least partially inserted therein into the body through the opening in the body wall with its front end forward and then separating the instrument from the member after the member and instrument are inserted into the body. Additionally, suture thread may be passed through a through opening in the front end of the member prior to inserting the member into the body so that after the instrument is separated from the member, the member is removable from the body by pulling the suture thread and urging the member out of the body through the opening with its front end forward. For the method, other ways to remove the member from the body are also envisioned.

Other and further objects, advantages and features of the present invention will be understood by reference to the following specification in conjunction with the annexed drawings, wherein like parts have been given like numbers.

### BRIEF DESCRIPTION OF THE DRAWINGS

The invention, together with further objects and advantages thereof, may best be understood by reference to the following description taken in conjunction with the accompanying drawings, wherein like reference numerals identify like elements, and wherein:

FIG. 1 is a side view of a device in accordance with the invention.

FIG. 2 is a cross-sectional view of the device taken along the line 2-2 of FIG. 1.

FIG. 3 is a rear perspective view of the device.

FIG. 4 is a view of the device showing a suture thread through an opening at the front end of the device.

FIG. 5 is a view showing a surgical stapler and the device in accordance with the invention prior to being coupled together.

FIG. 6 is a view showing the coupling of a surgical stapler and the device in accordance with the invention in a state in which it is ready for insertion into the body.

FIG. 7 is a view showing a surgical stapler anvil and the device in accordance with the invention prior to being coupled together.

FIG. 8 is a view showing the coupling of a surgical stapler anvil and the device in accordance with the invention in a state in which it is ready for insertion into the body.

### DETAILED DESCRIPTION OF THE INVENTION

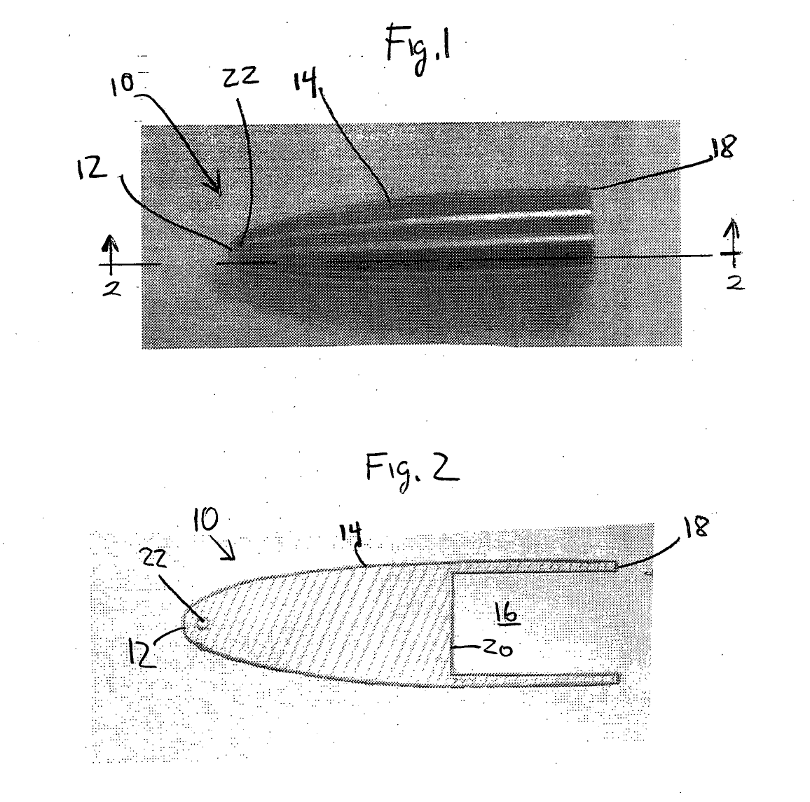

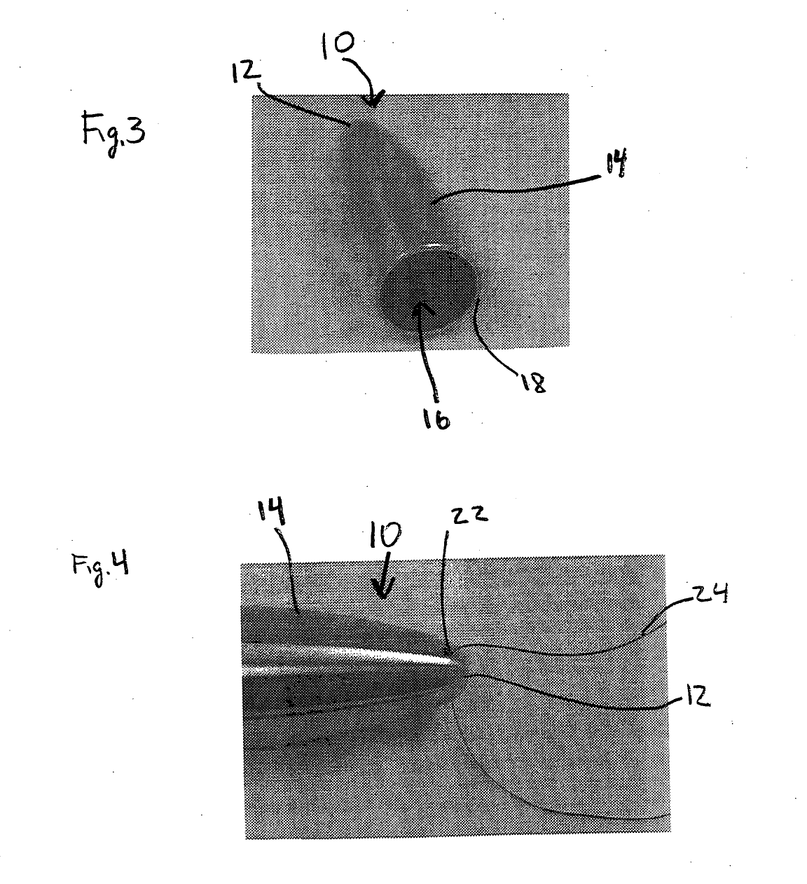

Referring to the accompanying drawings wherein like reference numerals refer to the same or similar elements, the device for enabling introduction of surgical instruments in accordance with the invention is designated generally as 10 and has the general shape of a cone or bullet with a rounded front end 12 and arcuate side surfaces 14. An internal cavity 16 extends inward from a rear edge 18 of the device 10 (see FIGS. 2 and 3) and is designed to accommodate at least part of a surgical instrument to be introduced into the body, such as a surgical stapler or stapler anvil. Cavity 16 may have a generally uniform cross-sectional shape and a substantially planar front wall 20 substantially perpendicular to a longitudinal axis of the device 16. Thus, one form of the cavity 16 may be substantially cylindrical.

A through hole 22 is formed at the front end 12 of the device 10 to enable suture thread 24 to pass therethrough, the purpose of which is explained below. The diameter of the through hole 22 may be selected as desired for particularly sized devices 10 and/or suture threads 24. The through hole 22 preferably defines an axis which is substantially perpendicular to the longitudinal axis of the device 10 such that pulling of the thread 24 results in an even application of force to the device 10 which is beneficial during removal of the device 10 from the body in the manner discussed below. The through hole 22 may be substantially cylindrical, and optionally has flanged openings at one or both ends to better accommodate the suture and facilitate insertion thereof.

Both the diameter and length of the introducing device 10, as well as the diameter and depth or length of the cavity 16, can vary depending on the size of the surgical instrument being introduced into the body. For example, several different sized introduction devices 10 can be produced, each designed for use with one or more commercial staplers or commercial stapler anvils. During surgery, the introduction device compatible with the stapler or anvil being used would then be selected and used in order to introduce the stapler or anvil into the patient. In addition, for abdominal surgical applications, longer devices 10 can be produced to accommodate various sizes (thicknesses) of the abdominal wall, e.g., a longer device would be used for obese patients than for thin patients. Another variation in the construction of the device 10 would be to change the tapering radius of the body 10 and/or front end 12 from that in the illustrated embodiment.

The device 10 may be formed as a unitary member or body and may be made from surgical grade stainless steel, if a reusable device is desired, or from high density polyethylene or plastic, if a single-use disposable device is desired. A device having the same dimensions can be formed from both stainless steel or polyethylene or plastic to provide the surgeon with options for use of the device.

For a reusable device, the device would be sterilized prior to each use by conventional methods used in the preparation of surgical instruments. For a disposable device, the device would be supplied in a sterile package and discarded after use.

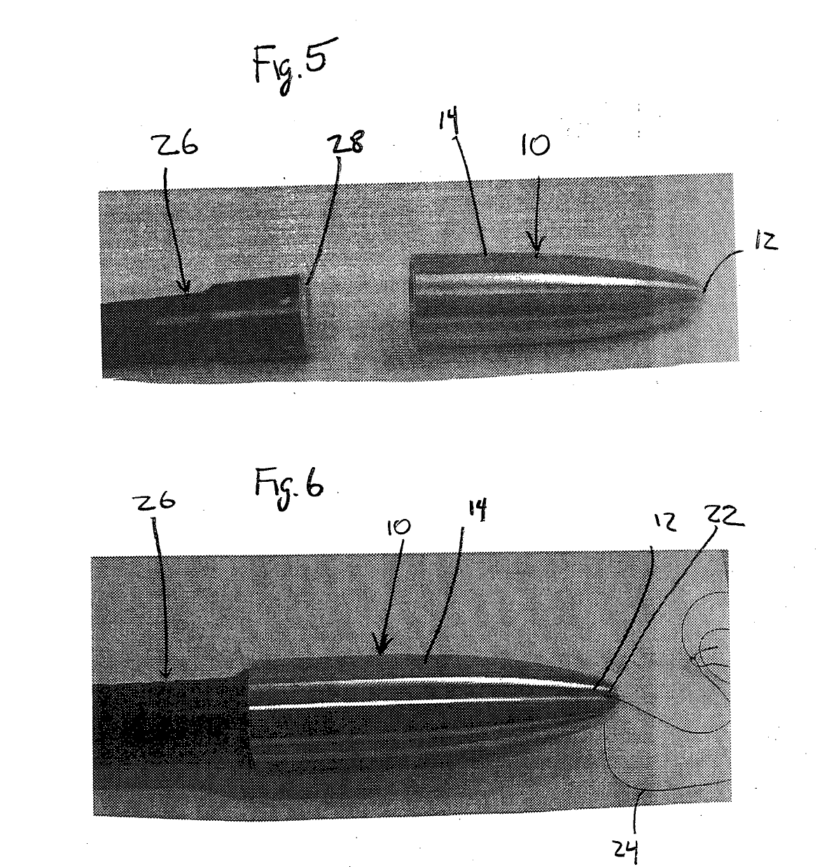

FIGS. 5 and 6 show the use of the device 10 in combination with a circular surgical stapler 26. As shown in FIG. 5, the stapler 26 has a blunt open forward end 28 which makes it difficult to surgically introduce the stapler through a small opening in the body wall and can also cause fragments of the tissues around the opening in the body wall to become lodged into the opening in the forward end 28 of the stapler 26. The tissue fragments could cause the stapling mechanism in the stapler 26 to malfunction.

As shown in FIG. 6, in preparation for use, at least the forward end 28 of the stapler 26 is inserted into the cavity 16 in the device 10 to thereby attach the stapler 26 to the device 10. Suture thread 24 is then inserted through the through hole 22 until it extends on both sides of the through hole. One end or both ends of the thread 24 would also be fixed or tied external to the patient or the ends of the thread 24 are tied together. The device 10 with attached stapler 26 is then inserted with its front end 12 forward through the opening in the body wall, e.g., a laparoscopic opening in the abdominal wall, while maintaining a portion of the thread 24 outside of the body. Insertion of the device 10 is facilitated by the rounded shape of the front end 12 of the device 10 and the smooth arcuate surfaces 14 which are effective to spread the tissues around the opening outward and enable smooth passage of the device 10 and stapler 26 therethrough.

Once in the abdominal cavity, the device 10 is removed from engagement with the stapler 26 by pulling on the suture thread 24. After removal of the device 10, the stapler 26 is situated in the abdominal cavity and ready for use. The forward end 28 of the stapler 26 has thus been prevented from receiving tissue fragments during its insertion into the abdominal cavity.

After disengagement from the stapler 26 and removal of the stapler 26 after use, the device 10 is removed from the abdominal cavity preferably with its front end 12 forward, for example, through the same opening through which the device 10 and stapler 26 were inserted into the abdominal cavity. Removal of the device 10 is facilitated by the rounded shape of the front end 12 of the device 10 which spreads the tissues around the opening outward and enable smooth passage of the device 10 therethrough.

The stapler 26 is also removed from the abdominal cavity after use in a conventional manner, i.e., through the same opening through which it was inserted into the patient.

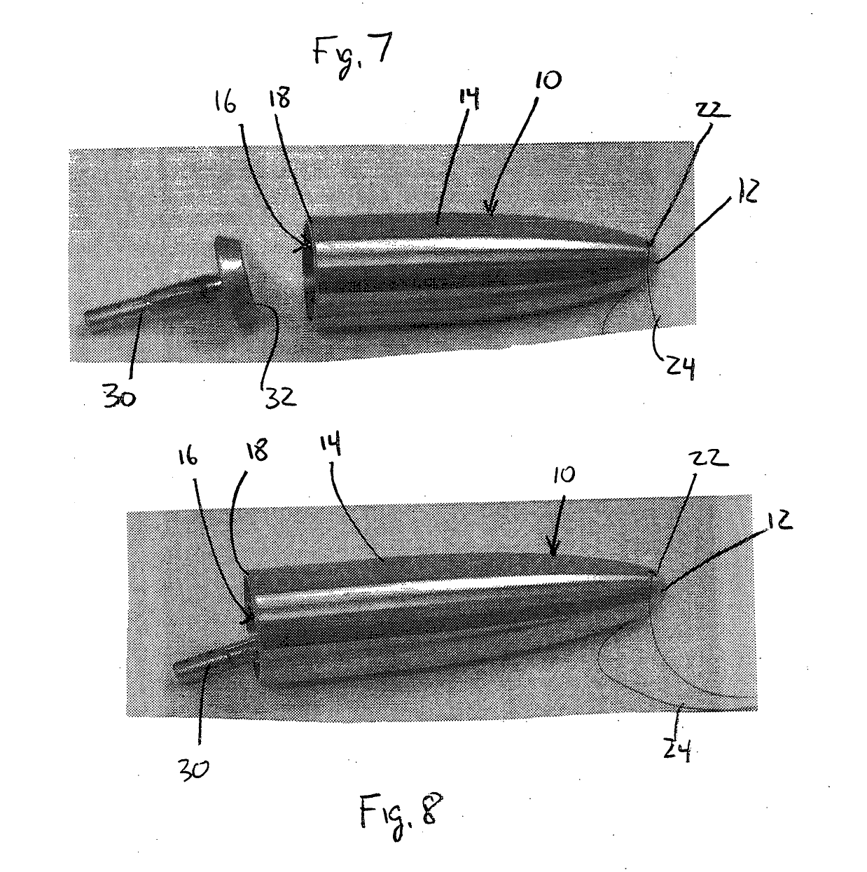

FIGS. 7 and 8 show the use of the device 10 in combination with a stapler anvil 30. The anvil 30 has a blunt forward end 32 (see FIG. 7) which makes it difficult to surgically introduce the anvil through a small opening in the body wall. As shown in FIG. 8, in preparation for use, at least the forward end 32 of the anvil 30 is inserted into the cavity 16 in the device 10. Suture thread 24 is then inserted through the through hole 22 and one end or both ends of the thread 24 are fixed or tied external to the patient or the ends of the thread 24 are tied together. The device 10 with the anvil 30 at least partially received therein, is then inserted through the opening in the body wall with its front end 12 forward, e.g., a laparoscopic opening in the abdominal wall, while maintaining a portion of the thread 24 outside of the body. Once in the abdominal cavity, the device 10 is removed from engagement with the anvil 30 by pulling on the suture thread 24. After removal of the device 10, the anvil 30 is situated in the abdominal cavity and ready for use.

After disengagement from the anvil 30, the device 10 is removed from the abdominal cavity with its front end 12 forward, for example, through the same opening through which the device 10 and anvil 30 were inserted into the abdominal cavity. The anvil 30 is also removed from the abdominal cavity after use in a conventional manner, i.e., through the same opening through which it was inserted into the patient.

Advantages of the use of the device 10 to insert surgical instruments into the body, such as a surgical stapler and stapler anvil, include a reduction in the time and effort required to introduce the surgical stapler or stapler anvil into the body, a reduction in the trauma caused to the tissue around the opening through which the surgical stapler or stapler anvil is inserted into the body, an improvement in the ergonomic position of the surgeon during the introduction of the surgical stapler or stapler anvil and the prevention of the entrapment of tissues of the body wall surrounding the opening with the stapler mechanism. In addition, the size of the opening in the body wall can be made quite small since the spreading action of the device 10 effectively, but only temporarily, expands the opening to allow the surgical stapler or stapler anvil to pass therethrough.

While the invention has been described above with respect to specific apparatus and specific implementations, it should be clear that various modifications and alterations can be made, and various features of one embodiment can be included in other embodiments, within the scope of the present invention. For example, although the invention is described above for use in connection with surgery on humans, it can be used for animals. It can also be used to facilitate the insertion of surgical instruments into any part of the body and is not limited to insertion of surgical staplers and stapler anvils into the abdominal cavity. Moreover, although sometimes described for use in connection with laparoscopic openings, the invention can be used to insert surgical instruments through other types of openings in the body or body wall.

1. A device for facilitating insertion of a surgical instrument into a human body through an opening in a body wall, comprising:
a cone-shaped member having a rounded front end, an internal cavity extending inward from a rear edge and a through opening arranged at said front end,
said cavity being receivable of at least part of the surgical instrument such that when received in said cavity, the surgical instrument is insertable into the body through the opening in the body wall by urging said member through the opening with said front end forward and thereby causing tissue surrounding the opening to spread outward.

2. The device for facilitating insertion of a surgical instrument into a human body through an opening in a body wall as defined by claim 1, wherein the instrument is separable from said member after insertion of said member and the instrument into the body.

3. The device for facilitating insertion of a surgical instrument into a human body through an opening in a body wall as defined by claim 1, wherein said cavity has a substantially uniform cross-sectional shape in a longitudinal direction.

4. The device for facilitating insertion of a surgical instrument into a human body through an opening in a body wall as defined by claim 1, wherein said cavity is defined by a substantially planar front wall oriented substantially perpendicular to a longitudinal axis of said member.

5. The device for facilitating insertion of a surgical instrument into a human body through an opening in a body wall as defined by claim 1, wherein said cavity is substantially cylindrical.

6. The device for facilitating insertion of a surgical instrument into a human body through an opening in a body wall as defined by claim 1, wherein said through hole defines an axis substantially perpendicular to a longitudinal axis of said member.

7. The device for facilitating insertion of a surgical instrument into a human body through an opening in a body wall as defined by claim 1, wherein said through hole is substantially cylindrical.

8. The device for facilitating insertion of a surgical instrument into a human body through an opening in a body wall as defined by claim 1, wherein said member is made from stainless steel.

9. The device for facilitating insertion of a surgical instrument into a human body through an opening in a body wall as defined by claim 1, wherein said member is made from high density polyethylene or plastic.

10. The device for facilitating insertion of a surgical instrument into a human body through an opening in a body wall as defined by claim 1, wherein said member has arcuate side surfaces extending between said rear edge and said front end.

11. A method for introducing a surgical instrument into a body through an opening in a body wall, comprising the steps of:
forming a cone-shaped member having a rounded front end and an internal cavity extending inward from a rear edge;
inserting at least part of the instrument into the cavity;
inserting the member with the instrument at least partially inserted therein into the body through the opening in the body wall with its front end forward; and
separating the instrument from the member after the member and instrument are inserted into the body.

12. The method for introducing a surgical instrument into a body through an opening in a body wall as defined by claim 11, further comprising the steps of:
forming a through opening at the front end of the member;
passing suture thread through the through opening prior to inserting the member into the body; and
after the instrument is separated from the member, removing the member from the body by pulling the suture thread and urging the member out of the body through the opening with its front end forward.

13. The method for introducing a surgical instrument into a body through an opening in a body wall as defined by claim 12, further comprising the steps of:
tying ends of the suture thread together to form a loop prior to inserting the member into the body; and
maintaining a part of the loop of the suture thread outside of the body during the insertion of the member into the body.

14. The method for introducing a surgical instrument into a body through an opening in a body wall as defined by claim 11, wherein the surgical instrument is a stapler having a blunt open forward end which is inserted into the cavity in the member.

15. The method for introducing a surgical instrument into a body through an opening in a body wall as defined by claim 11, wherein the surgical instrument is an anvil having a blunt forward end which is inserted into the cavity in the member.
